(19) United States
(12) Patent Application Publication
(10) Pub. No.: US 2007/0166338 A1
(43) Pub. Date: Jul. 19, 2007

(54) Dermabrasion composition and process

(76) Inventor: Reilly Dillon, Chanhassen, MN (US)

Correspondence Address: BRIGGS AND MORGAN P.A., 2200 IDS CENTER, 80 SOUTH 8TH ST, MINNEAPOLIS, MN, 55402, US

(21) Appl. No.: 11/332,038

(22) Filed: Jan. 13, 2006

(51) Int. Cl. A61K 8/73

(52) U.S. Cl. 424/401; 424/70.13

(57) ABSTRACT

A composition of matter for abrading the epidermis of a human body to expose the underlying dermis, consisting of: (a) an abrasive; (b) a dessicant; and (c) a thickening agent (d) in the absence of an emollient in an aqueous medium. A method of removing the epidermis of a human body to expose the underlying dermis, comprising the steps of: (a) applying to the epidermis a composition of matter comprising an abrasive, a dessicant, and a thickening agent; (b) evaporating the dessicant from the skin, leaving the abrasive on the dessicated epidermis; (c) applying mechanical force to the dessicated epidermis, thereby removing the epidermis; and d) rinsing the remaining dermis.

### BACKGROUND OF THE INVENTION

This invention relates to a topical skin treatment composition in the form of a cosmetic composition using an improved method for revitalizing and retexturing the skin, especially by peeling the outer layer of micro thin dead skin cells, sebum, and other impurities that are compacted regularly onto the stratum cornea as part of normal metabolism.

The layers of skin consist of the epidermis, the dermis and the subcutaneous tissue. The outermost layer, known as the dermis, itself consists of four layers, the stratum cornea, the stratum granulose, the squalors cell layer, and the basal cell layer. Through a process known as desquamation, the epidermis has the ability to constantly renew itself by shedding the flattened dead skin cells composing the stratum cornea. However, over time this normal metabolic process slows down, calling for skin compositions that effectively peel, exfoliate and remove these dead cells and related impurities, accelerating desquamation that is a part of the natural cell renewal cycle of the skin.

The stratum corneum is the outer coating of the epidermis. It consists of compacted dead cells called keratinocytes that are composed of keratin, a dried out protein. This is the layer of skin that is felt and seen and can be characterized as for example, smooth, soft, oily or flaky. Cells of the basal cell layer take about two to four weeks to migrate upwards through the four epidermal layers until they reach the top layer, the stratum corneum. There they are compacted and under optimal circumstances, shed over a period of two to six weeks. Thus, the usual turnover time of the epidermis, namely the time it takes for cells to migrate from the basal cell layer to the point of being shed from the stratum corneum, is about four to eight weeks. However, although the stratum corneum is a multicellular membrane of metabolically active cells, it begins to lose its dynamic ability to constantly renew itself through desquamation with the natural progression of the aging process. Young skin renews its surface layers every 2 to 3 weeks, whereas mature skin may take twice as along to be renewed as compared to young skin. If we accelerate the process by which the rate of dead skin cells in the stratum corneum are being desquamated, then it follows that the rate at which new cells are formed of epidermal tissues, restoring the skin's freshness and youthful appearance.

Periodically, the facial skin of the human body needs a deep cleansing to remove not only dead skin generated by epidermal desquamation, but also the oily particles excreted from oil glands. The second layer of skin or dermis, houses the sebaceous glands. These oil glands continuously excrete and channel oil from the deep dermis to minute openings at the surface of the skin. This natural skin oil, called sebum, has a tendency to solidify over the sebaceous orifice resulting in the formation of hardened plaques. Such plaques contribute to the growth of adverse dermatologic skin conditions such as acne vulgaris, cysts, white and black heads.

The disease of acne vulgaris is reported to be the singly most common skin disease and affects approximately eighty percent of the teenage population. However, it may persist into the third and fourth decades of life. Acne primarily is a disease of the pilosebaceous system with a multifactorial cause. The pathogenesis includes an androgen dependent increase in sebum production, proliferation of the follicular microflora (principally P. acnes) and alteration in the follicular keratinization. This results in the primary clinical lesions of acnes, namely, the open comedone (blackheads), closed comedone (whiteheads), papules, pustules and nodules. The increased sebum production is responsible for the oily appearance. Currently, therapy is directed towards treatment of the lesions. The presence of oil itself is not the cause of acne but is a great psychological problem for the acne patient.

The onset of acne vulgaris is related to adolescence and normal sexual and physical growth. During this rapid linear growth period there is a marked development in the pilosebaceous system which results in sebum production and changes in its composition and physical characteristics. These events are hormonally controlled.

The disease of the pilosebaceous follicle is first detectable by change occurring in the follicular epithelium. The pilosebaceous unit is made up of a hair follicle and a pilosebaceous gland which are connected to the skin surface ducts through which the hair passes. The sebaceous gland produces sebum which is a mixture of fats and waxes that transgress the duct and spread to the skin surface which helps keep the skin soft and moist. The acne lesion develops when the gland and lining begin to work excessively which predominantly occurs during puberty. The glands produce more sebum making the skin oily. The duct normally sheds cells which are carried to the skin surface by the sebum. When acne develops, cells stick together to form a thick layer and plug the duct. More cells and sebum pile up behind this plug which results in the primary lesion of acne, the comedone. If the plug stays below the skin surface, it is called a “closed comedone” or a “whitehead.” A comedone which pushes through the surface is referred to as an “open comedone” or “blackhead.” This is not due to dirt but due to discoloration of melanin, the dark pigment in normal skin. The whiteheads and blackheads are referred to a “noninflammatory acne lesions.” However, the pilosebaceous unit can rupture and become inflamed and these are the pimples, papules, and pustules which are the inflammatory lesions of this disease.

Pilosebaceous units are found all over the body, but they are more predominant on the face, chest, and the back. These are usually the predominant areas which develop acne.

The existence of these plaques in combination with the constant drying of the outermost epidermal cells contributes to the appearance of rough, aged, and unhealthy skin.

There are numerous skin care preparations on the market today which have been designed to promote healthy and youthful-looking skin. Among various preparations include emollients or occlusive agents which moisturize the skin and prevent dryness. Other skin preparations have been formulated to exhibit antiseptic and astringent properties, while still other preparations are used as skin cleansers that attempt to exfoliate the skin. However, none of such preparations adequately remove the epidermis without irritation to the patient.

U.S. Pat. No. 6,036,966 discloses skin treatment compositions comprising protein and enzyme abstracts. While this patent discloses the use of slightly abrasive material, it also requires the use of unoxidized nerve tissue and an emollient or lubricating vehicle to help hydrate the skin, rather than volatile vehicles, such as ethanol, that dry the skin. In contrast, the present invention does not require unoxidized nerve tissue and is specifically designed to use a dessicant, such as ethanol, to remove moisture from the skin.

### SUMMARY OF THE INVENTION

A composition of matter for abrading the epidermis of a human body to expose the underlying dermis, the composition comprising:
(a) an abrasive;
(b) a dessicant; and
(c) a thickening agent
(d) in the absence of an emollient
   in an aqueous medium.

A method of removing the epidermis of a human body to expose the underlying dermis, comprising the steps of:
(a) applying to the epidermis a composition of matter comprising an abrasive, a dessicant, and a thickening agent;
(b) evaporating the dessicant from the skin, leaving the abrasive on the dessicated epidermis;
(c) applying mechanical force to the dessicated epidermis, thereby removing the epidermis; and
d) rinsing the remaining dermis.

A principle object and advantage of the present invention is that it thoroughly removes the epidermis from the dermis by using a combination of a dessicant and an abrasive.

Another object and advantage of the present invention is that the epidermis can be removed with minimal experience and instruction without significant damage or destruction to the dermis and at low cost.

### BRIEF DESCRIPTION OF THE DRAWINGS

FIGS. 1A-1E illustrate the steps of the claimed method.

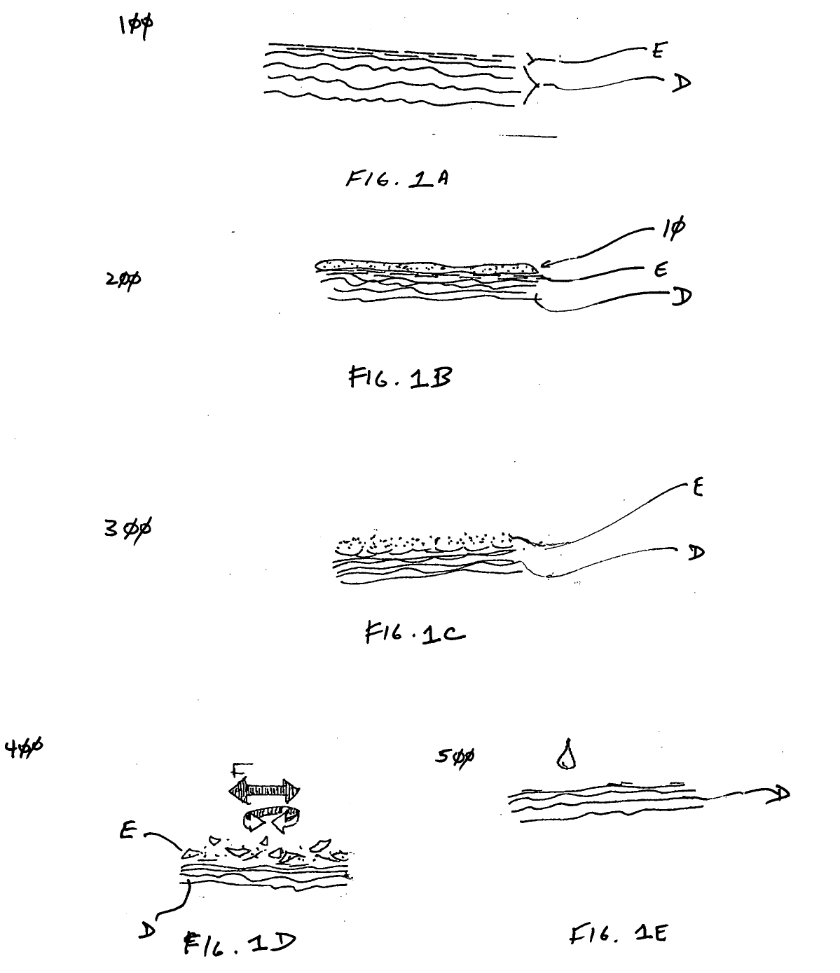

### DETAILED DESCRIPTION OF THE PREFERRED EMBODIMENT

In one aspect, the present invention is a composition of matter 10 for abrading the epidermis of a human body to expose the underlying dermis, the composition comprising: an abrasive; a dessicant; and a thickening agent in the absence of an emollient in an aqueous medium.

Preferably, the abrasive is selected from the group consisting of sodium bicarbonate, aluminum oxide, and sodium chloride. However, any similar abrasive may be used, such as, without limitation, inorganic powders, such as calcium carbonate, acetate, formate, gluconate, lactate, oxalate, phosphate or stannate; magnesium carbonate; zinc carbonate; magnesium aluminum silicate; silica; zinc aluminum silicate; talc; kaolin; cericite; mica; vermiculite; diatomaceous earth; calcium silicate; barium silicate; barium sulfate; hydroxyapatite; zeolite; and boran nitride.

Preferably, the dessicant is selected from the group consisting of acetone and an alcohol. However, other well-known dessicants may also be used. Most preferably, the alcohol is selected from the group consisting of isopropyl alcohol and ethyl alcohol.

Preferably, the thickening agent is selected from the group consisting of xanthan gum, gum Arabic, and guar gum, and compositions thereof. Most preferably, the thickening agent is a commercial food thickener known as Simply Thick™, which is composed of xanthum gum, citric acid, potassium sorbate, sodium benzoate, and water. However, other well-known thickening agents may also be used.

In the preferred embodiment, the abrasive is present in about 40-50%; the dessicant is present in about 40-50% and in turn comprises about 70-100% isopropyl alcohol and 50-100% ethyl alcohol; and thickening agent is present in about 1-20%.

In one aspect, the present invention is a method of removing the epidermis E of a human body to expose the underlying dermis D, comprising the steps of: 200—applying to the epidermis E a composition of matter 10 comprising an abrasive, a dessicant, and a thickening agent; 300—evaporating the dessicant from the skin, leaving the abrasive on the dessicated epidermis E; 400—applying mechanical force to the dessicated epidermis E, thereby removing the epidermis; and 500—rinsing the remaining dermis D.

Preferably, the composition of matter excludes an emollient.

Preferably, the abrasive is selected from the group consisting of sodium bicarbonate, aluminum oxide, and sodium chloride. However, any similar abrasive may be used, such as, without limitation, inorganic powders, such as calcium carbonate, acetate, formate, gluconate, lactate, oxalate, phosphate or stannate; magnesium carbonate; zinc carbonate; magnesium aluminum silicate; silica; zinc aluminum silicate; talc; kaolin; cericite; mica; vermiculite; diatomaceous earth; calcium silicate; barium silicate; barium sulfate; hydroxyapatite; zeolite; and boran nitride.

Preferably, the dessicant is selected from the group consisting of acetone and an alcohol. However, other well-known dessicants may also be used. Most preferably, the alcohol is selected from the group consisting of isopropyl alcohol and ethyl alcohol.

Preferably, the thickening agent is selected from the group consisting of xanthan gum, gum Arabic, and guar gum, and compositions thereof. Most preferably, the thickening agent is a commercial food thickener known as Simply Thick™, which is composed of xanthum gum, citric acid, potassium sorbate, sodium benzoate, and water. However, other well-known thickening agents may also be used.

In the preferred embodiment, the abrasive is present in about 40-50%; the dessicant is present in about 40-50% and in turn comprises about 70-100% isopropyl alcohol and 50-100% ethyl alcohol; and thickening agent is present in about 1-20%.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described below. All publications, patent applications, patents, and other references mentioned herein are incorporated by reference in their entirety to the extent allowed by applicable law and regulations. In case of conflict, the present specification, including definitions, will control.

The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention.

What is claimed:

1. A composition of matter for abrading the epidermis of a human body to expose the underlying dermis, the composition comprising:
(a) an abrasive;
(b) a dessicant; and
(c) a thickening agent
(d) in the absence of an emollient
in an aqueous medium.

2. The composition of matter of claim 1, wherein the abrasive is selected from the group consisting of sodium bicarbonate, aluminum oxide, and sodium chloride.

3. The composition of matter of claim 1, wherein the dessicant is selected from the group consisting of acetone and an alcohol.

4. The composition of matter of claim 3, wherein the alcohol is selected from the group consisting of isopropyl alcohol and ethyl alcohol.

5. The composition of matter of claim 1, wherein the thickening agent is selected from the group consisting of: xanthan gum, gum Arabic, and guar gum and compositions thereof.

6. The composition of matter of claim 5, wherein the thickening agent comprises a commercial food thickener further comprising a mixture of xanthum gum, citric acid, potassium sorbate, sodium benzoate, and water.

7. The composition of matter of claim 1, further comprising about 40-50% abrasive, about 40-50% dessicant, about 1-20% thickening agent and the remainder water.

8. The composition of matter of claim 8, wherein the dessicant further comprises about 70-100% isopropyl alcohol and about 50-100% ethyl alcohol.

9. A composition of matter for abrading the epidermis of a human body to expose the underlying dermis, the composition consisting essentially of:
(a) an abrasive;
(b) a dessicant; and
(c) a thickening agent.

10. The composition of matter of claim 8, wherein the abrasive is selected from the group consisting of sodium bicarbonate, aluminum oxide, and sodium chloride.

11. The composition of matter of claim 8, wherein the dessicant is selected from the group consisting of acetone and an alcohol.

12. The composition of matter of claim 10, wherein the alcohol is selected from the group consisting of isopropyl alcohol and ethyl alcohol.

13. The composition of matter of claim 8, wherein the thickening agent is selected from the group consisting of: xanthan gum, gum Arabic, and guar gum and compositions thereof.

14. The composition of matter of claim 12, wherein the thickening agent comprises a commercial food thickener further comprising a mixture of xanthum gum, citric acid, potassium sorbate, sodium benzoate, and water.

15. The composition of matter of claim 8, further comprising about 40-50% abrasive, about 40-50% dessicant, about 1-20% thickening agent and the remainder water.

16. The composition of matter of claim 15, wherein the dessicant further comprises about 70-100% isopropyl alcohol and about 50-100% ethyl alcohol.

17. A method of removing the epidermis of a human body to expose the underlying dermis, comprising the steps of:
(a) applying to the epidermis a composition of matter comprising an abrasive, a dessicant, and a thickening agent;
(b) evaporating the dessicant from the skin, leaving the abrasive on the dessicated epidermis;
(c) applying mechanical force to the dessicated epidermis, thereby removing the epidermis; and
(d) rinsing the remaining dermis.

18. The method of claim 15, wherein the composition of matter excludes an emollient.

19. The method of claim 15, wherein the abrasive is selected from the group consisting of sodium bicarbonate, aluminum oxide, and sodium chloride.

20. The method of claim 15, wherein the dessicant is selected from the group consisting of acetone and an alcohol.

21. The method of claim 18, wherein the alcohol is selected from the group consisting of isopropyl alcohol and ethyl alcohol.

22. The method of claim 15, wherein the thickening agent is selected from the group consisting of: xanthan gum, gum Arabic, and guar gum and compositions thereof.

23. The method of claim 20, wherein the thickening agent comprises a commercial food thickener further comprising a mixture of xanthum gum, citric acid, potassium sorbate, sodium benzoate, and water.

24. The composition of matter of claim 15, further comprising about 40-50% abrasive, about 40-50% dessicant, about 1-20% thickening agent and the remainder water.

25. The composition of matter of claim 24, wherein the dessicant further comprises about 70-100% isopropyl alcohol and about 50-100% ethyl alcohol
